(19) United States
(12) Patent Application Publication
(10) Pub. No.: US 2018/0325440 A1
(43) Pub. Date: Nov. 15, 2018

(54) EMOTION REGULATION DEVICE, WEARABLE DEVICE AND CAP FOR RELIEVING EMOTION

(71) Applicants: BOE TECHNOLOGY GROUP CO., LTD., Beijing (CN); BOE OPTICAL SCIENCE AND TECHNOLOGY CO., LTD., Suzhou, Jiangsu (CN)

(72) Inventor: Hongyun WANG, Beijing (CN)

(21) Appl. No.: 15/512,830

(22) PCT Filed: May 17, 2016
(86) PCT No.: PCT/CN2016/082330

§ 371 Date: Mar. 20, 2017

(30) Foreign Application Priority Data
Feb. 19, 2016 (CN) 201610094334.9

(51) Int. Cl. A61B 5/16; A61B 5/0205; A61M 21/00

(52) U.S. Cl. CPC A61B 5/165; A61B 5/11; A61M 21/00; A61B 5/0205

(57) ABSTRACT

The present disclosure relates to an emotion regulation device, a wearable device and a cap for relieving emotion. The emotion regulation device comprises: at least one detection component for obtaining a current physiological parameter value and/or behavior data of a user in real time; a processing component for determining a current emotional state of the user based on the current physiological parameter value and/or the behaviour data, selecting an appropriate execution component based on the current emotional state, and sending a control instruction to the selected execution component; and at least one execution component for executing a corresponding control action based on the control instruction.

### TECHNICAL FIELD

The present disclosure relates to the technical field of electronic devices, particular to an emotion regulation device, a wearable device and a cap for relieving emotion.

### BACKGROUND

The social and economic development becomes faster and faster. With the acceleration of the pace of life, work task becomes more and more heavy. People are under increasing pressure and become more and more nervous. In this case, more and more people suffer from a variety of psychological or physiological barriers and diseases, e.g., psychological sub-health, depression, anxiety neurosis and pseudoinsomnia etc. These psychological barriers and diseases have become a big social problem.

For patients suffering from depression, not only the patients' own mind and body suffer from the disease, relatives of the patients also suffer a lot from it. Because such patients themselves are passive to treatment of the disease, it is difficult for them to communicate with friends and family members although they feel very sad. If the bad state cannot be alleviated for a long time, accident may occur easily. Hence, relatives of the patients must stay with the patients at all times so as to prevent occurrence of accident. In addition, for people with a history of depression, even if they have recovered, it is difficult to determine whether the disease will relapse or when it relapses. Hence, even if they are cured, relatives of the patients also do not rest assured to let the patients live alone. The anxiety neurosis is also likely to turn into depression under external stimuli.

Therefore, how to identify emotions of the anxiety neurosis or depression patients and adopt corresponding emotion relieving measures has become a problem that has to be solved urgently at present.

### SUMMARY

The present disclosure provides an emotion regulation device, a wearable device and a cap for relieving emotion, so as to learn the current emotional state of a user, thereby regulating the abnormal emotional state of the user timely.

Embodiments of the present disclosure provide an emotion regulation device, comprising: at least one detection component for obtaining a current physiological parameter value and/or behavior data of a user in real time; a processing component for determining a current emotional state of the user based on the current physiological parameter value and/or the behaviour data, selecting an appropriate execution component based on the current emotional state, and sending a control instruction to the selected execution component; as well as at least one execution component for executing a corresponding control action based on the control instruction.

In one embodiment, the detection component is used for: obtaining a parameter value for at least one physiological activity of voice, heart rate, blood pressure, body temperature and brainwave of a user as the current physiological parameter value, and/or obtaining data for at least one behavior of facial expression, gesture and pace of the user as the bevavior data. In this embodiment, different physiological activities or behaviors of the user can be monitored in real time respectively, so as to obtain a detailed data that can reflect the current emotional state of the user timely.

In one embodiment, the detection component comprises a physiological detection component and/or a behavior detection component. The physiological detection component comprises at least one of a sound sensor, a heart rate detector, a blood pressure sensor, a body temperature sensor and a brain scanner. The behavior detection component comprises at least one of a camera and a limb sensor.

In one embodiment, the processing component is used for: analyzing the current physiological parameter value and/or the behavior data, and determining one of a happy state, a mild anxiety state, a moderate anxiety state, a mild depressive state and a moderate depressive state as the current emotional state.

After the current emotional state is determined as the happy state, an execution component for recording is selected and a control instruction for recording an object, a sound and a scene that the user contacts is sent to the selected execution component for recording.

After the current emotional state is determined as the mild anxiety state or the mild depressive state, an execution component for relieving emotion is selected and a control instruction for relieving user emotion is sent to the selected execution component for relieving emotion.

After the current emotional state is determined as the moderate anxiety state or the moderate depressive state, an execution component for relieving emotion and an execution component for alarming are selected, and a control instruction for relieving user emotion is sent to the selected execution component for relieving emotion and a control instruction for alarming is sent to the selected execution component for alarming.

In one embodiment, the processing component is further used for, after the current emotional state of the user is determined as the mild anxiety state and does not change after a duration greater than or equal to a first time threshold, selecting an execution component for alarming and sending a control instruction for alarming to the selected execution component for alarming.

In one embodiment, the processing component is further used for, after the current emotional state of the user is determined as the mild depressive state and does not change after a duration greater than or equal to a second time threshold, selecting an execution component for alarming and sending a control instruction for alarming to the selected execution component for alarming.

In one embodiment, the execution component for recording comprises a sound recording device and/or an image recording device. The execution component for relieving emotion comprises at least one of a music player with earphones, smart glasses capable of playing videos, a display device, and a projector device. The execution component for alarming comprises at least one of a wireless transmitting device, a sound alarm, and a signal indicator lamp.

In one embodiment, the processing component is further used for sending the object, sound and scene that the user contacts when the user emotional state is the happy state recorded by the execution component for recording to the execution component for relieving emotion.

In one embodiment, the emotion regulation device further comprises: a storage component for storing the current physiological parameter value and/or the behavior data of the user obtained by the detection component; and a power supply component for supplying power for the processing component, the detection component, the execution component and the storage component.

In one embodiment, the storage component is further used for storing the object, sound and scene that the user contacts when the the user emotional state is the happy state recorded by the execution component for recording. The execution component for relieving emotion is further used for calling the object, sound and scene that the user contacts when the user emotional state is the happy state from the storage component.

The detection component monitors the current physiological state and/or behavior of the user in real time, and the processing component analyzes the data obtained by the detection component to determine a current emotional state of the user. In this way, appropriate execution components can be selected based on different emotional states of the patient to perform corresponding response actions, thereby realizing regulation of abnormal emotional states of the patient.

Embodiments of the present disclosure further provide a wearable device. The wearable device comprises a device body and an external accessory connected with the device body. The wearable device further comprises the emotion regulation device provided by any of the above embodiments.

In one embodiment, the detection component, the processing component and the execution component are selectively arranged within the device body and on the external accessory.

In one embodiment, the emotion regulation device further comprises a storage component and a power supply component. The storage component and the power supply component are arranged within the device body.

The device body and the external accessory of the wearable device can be provided with an emotion regulation device selectively, so as to be convenient for the user to carry. The detection component of the emotion regulation device monitors the current physiological state and/or behavior of the user in real time. The processing component analyzes the data obtained by the detection component to determine a current emotional state of the user. In this way, appropriate execution components can be selected based on different emotional states of the patient to perform corresponding response actions, thereby realizing regulation of abnormal emotional states of the patient.

Embodiments of the present invention further provide a cap for relieving emotion, comprising a cap body, a cap edge, and the emotion regulation device provided by any of the above embodiments.

In one embodiment, the detection component comprises a physiological detection component arranged within the cap body or on the cap edge and/or a behavior detection component arranged outside the cap body or on the cap edge. The physiological detection component comprises at least one of a sound sensor, a heart rate detector, a blood pressure sensor, a body temperature sensor and a brain scanner. The behavior detection component comprises at least one of a camera and a limb sensor.

In one embodiment, the execution component comprises: at least one of a sound recording device, a wireless transmitting device, a sound alarm and a music player arranged within the cap body, wherein the music player has earphones arranged outside the cap body; and/or, smart glasses with playing function arranged on the cap edge; and/or, at least one of an image recording device, a projector device and a signal indicator lamp arranged outside the cap body.

In one embodiment, the emotion regulation device further comprises a storage component and a power supply component arranged within the cap body.

The cap for relieving emotion is provided with an emotion regulation device, and the cap is convenient for the user to wear. The detection component of the emotion regulation device monitors the current physiological state and/or behavior of the user in real time. The processing component analyzes the data obtained by the detection component to determine a current emotional state of the user. In this way, appropriate execution components can be selected based on different emotional states of the patient to perform corresponding response actions, thereby realizing regulation of abnormal emotional states of the patient.

### BRIEF DESCRIPTION OF THE DRAWINGS

FIG. 1 is a structural block diagram of an emotion regulation device according to one embodiment of the present disclosure;

FIG. 2 is a specific structural block diagram of an emotion regulation device according to one embodiment of the present disclosure;

FIG. 3 is a structural block diagram of a wearable device according to one embodiment of the present disclosure;

FIG. 4 is a structural schematic view of a cap for relieving emotion according to one embodiment of the present disclosure.

### DETAILED DESCRIPTION OF THE EMBODIMENTS

Next, implementations of respective embodiments will be explained in detail with reference to the drawings of the description. It should be noted that the same or similar reference signs represent the same or similar elements or elements with the same or similar functions throughout the whole description. The embodiments described below with reference to the drawings are exemplary, which are only used for explaining the present disclosure and could not be understood as limitations to the present disclosure.

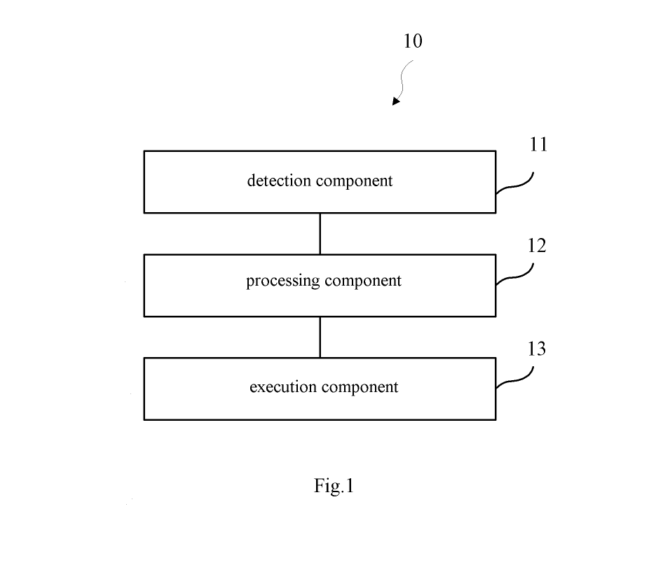

Referring to FIG. 1, embodiments of the present disclosure provide an emotion regulation device 10, comprising a processing component 12, at least one detection component 11 and at least one execution component 13.

The detection component 11 is used for obtaining a current physiological parameter value and/or behavior data of a user in real time.

The processing component 12 is used for determining a current emotional state of the user based on the current physiological parameter value and/or the behaviour data, selecting an appropriate execution component 13 based on the current emotional state, and sending a control instruction to the selected execution component 13.

The execution component 13 is used for executing a corresponding control action based on the control instruction.

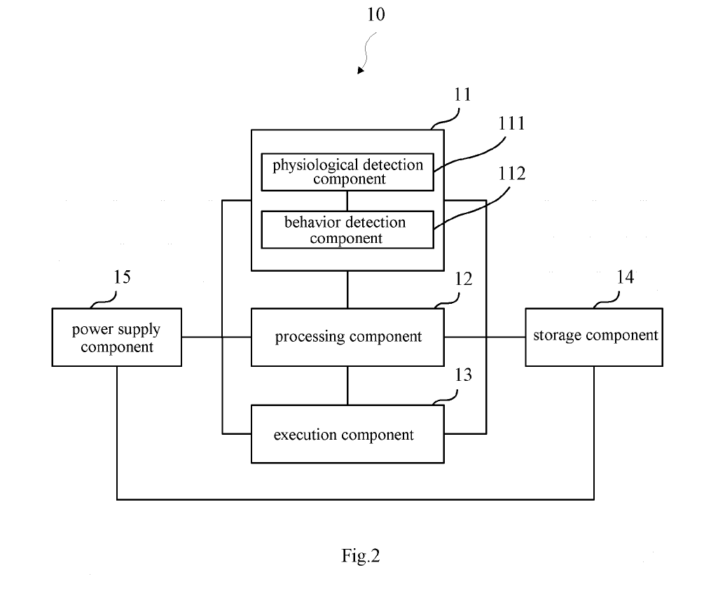

Certainly, in the emotion regulation device 10 provided by this embodiment, based on the consideration of different monitoring parameters or relatively complex emotion state regulation, the respective components have more specific functions and can also have more sub-portions. Next, explanation will be made by referring to FIG. 2.

The detection component 11 can be used for: obtaining a parameter value for at least one physiological activity of voice, heart rate, blood pressure, body temperature and brainwave of a user as the current physiological parameter value, and/or obtaining data for at least one behavior of facial expression, gesture and pace of the user as the bevavior data. The detection component 11 can monitor different physiological activities or behaviors of the user in real time respectively, so as to obtain the detailed data that can reflect the current emotional state of the user timely.

The detection component 11 can comprise a physiological detection component 111 and/or a behavior detection component 112. The physiological detection component comprises at least one of a sound sensor, a heart rate detector, a blood pressure sensor, a body temperature sensor and a brain scanner. The behavior detection component comprises at least one of a camera and a limb sensor.

The processing component 12 can be used for analyzing the current physiological parameter value and/or the behavior data, and determining one of a happy state, a mild anxiety state, a moderate anxiety state, a mild depressive state and a moderate depressive state as the current emotional state.

After the current emotional state is determined as the happy state, an execution component 13 for recording is selected, and a control instruction for recording an object, a sound and a scene that the user contacts is sent to the selected execution component 13 for recording.

After the current emotional state is determined as the mild anxiety state or the mild depressive state, an execution component 13 for relieving emotion is selected, and a control instruction for relieving user emotion is sent to the selected execution component 13 for relieving emotion.

After the current emotional state is determined as the moderate anxiety state or the moderate depressive state, an execution component 13 for relieving emotion and an execution component 13 for alarming are selected, and a control instruction for relieving user emotion is sent to the selected execution component 13 for relieving emotion and a control instruction for alarming is sent to the selected execution component 13 for alarming.

The processing component 12 can be further used for, after the current emotional state of the user is determined as the mild anxiety state and does not change after a duration greater than or equal to a first time threshold, selecting an execution component 13 for alarming and sending a control instruction for alarming to the selected execution component 13 for alarming.

the processing component can be further used for, after the current emotional state of the user is determined as the mild depressive state and does not change after a duration greater than or equal to a second time threshold, selecting an execution component 13 for alarming and sending a control instruction for alarming to the selected execution component 13 for alarming.

The above first time threshold and second time threshold can be set based on actual situations. For example, it can be set to be any time length from 1 minute to 3 hours. Specifically, the first time threshold is set to be 30 minutes, and the second time threshold is 1 hour. Certainly, the first time threshold and the second time threshold as examples only aim to explaining the present disclosure, rather than limiting the present disclosure.

The execution component 13 for recoding can comprise a sound recording device or an image recording device.

The execution component 13 for relieving emotion can comprise at least one of a music player with earphones, smart glasses capable of playing videos, a display device, and a projector device.

The execution component 13 for alarming can comprise at least one of a wireless transmitting device, a sound alarm, and a signal indicator lamp.

In some embodiments, the processing component can be further used for sending the object, sound and scene that the user contacts when the user emotional state is the happy state recorded by the execution component for recording to the execution component for relieving emotion. In other words, the object, sound and scene that the user contacts when the user emotional state is the happy state recorded by the execution component for recording can be displayed to the user by the execution component for relieving emotion, when the user emotional state is not good (such as in anxiety state or depressive state), so as to enable the user to relieve bad emotions through these beautiful sense and memory. These objects, sounds and scenes can be sent to the execution component for relieving emotion by the processing component directly and stored by the execution component for relieving emotion. They can also be stored by the processing component and sent to the execution component for relieving emotion when the processing component sends instructions to the execution component for relieving emotion. Certainly, in addition to the object, sound and scene recorded by the execution component for recording, the processing component or the execution component for relieving emotion can also store other objects, sounds and scenes that enable the user to feel better.

The emotion regulation device can further comprise a storage component 14. The storage component 14 is used for storing the current physiological parameter value and/or the behavior data of the user obtained by the detection component.

The emotion regulation device can further comprise a power supply component 15. The power supply component 15 can supply power for the processing component 12, the detection component 11, the execution component 13 and the storage component 14.

In some embodiments, the storage component can further store the object, sound and scene that the user contacts when the the user emotional state is the happy state recorded by the execution component for recording.

The execution component for relieving emotion can be further used for calling the object, sound and scene that the user contacts when the user emotional state is the happy state from the storage component.

That is to say, the object, sound and scene that the user contacts when the user emotional state is the happy state recorded by the execution component for recording is stored into a separate storage component. When the processing component sends an instruction for relieving the user emotion to the execution component for relieving emotion, the object, sound and scene that can relieve the user emotion in the storage component is called. These objects, sounds and scenes that can relieve the user emotion not only include the content recorded by the execution component for recording, but also can include other objects, sounds, and scenes stored in the storage component that enable the user to feel better.

In order to enable the emotion regulation device 10 to be portable, the emotion regulation device 10 can be combined with a wearable object, for example, cap, clothes etc.

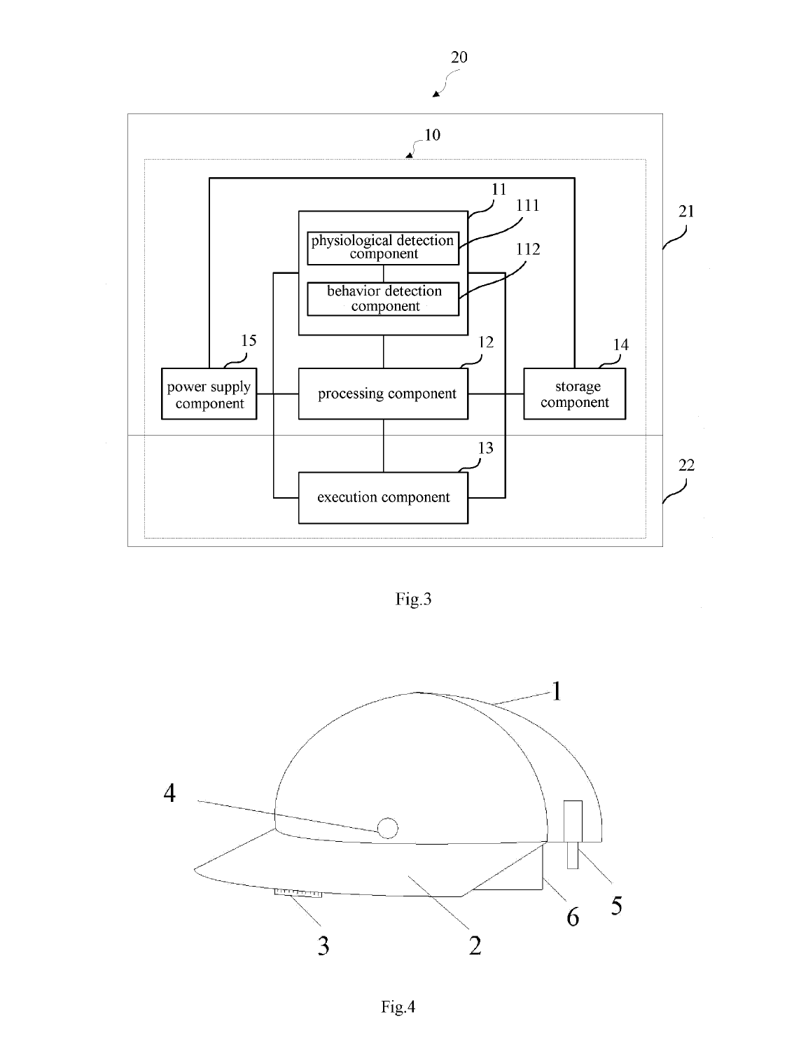

Referring to FIG. 3, embodiments of the present disclosure provide a wearable device 20, comprising a device body 21 and an external accessory 22 connected with the device body 21. The wearable device 20 further comprises the emotion regulation device 10 provided by any of the above embodiments.

The detection component 11, the processing component 12 and the execution component 13 of the emotion regulation device 10 are selectively arranged within the device body 21 and on the external accessory 22.

The emotion regulation device 10 can further comprise a storage component 14 and a power supply component 15. The storage component 14 and the power supply component 15 are arranged within the device body 21.

Referring to FIG. 4, based on a same inventive concept, embodiments of the present disclosure further provide a cap for relieving emotion. The cap comprises a cap body 1, a cap edge 2, and the emotion regulation device provided by any of the above embodiments. The emotion regulation device 10 is as shown in FIGS. 1 and 2.

The detection component 11 of the emotion regulation device 10 can comprise a physiological detection component 111 arranged within the cap body 1 or on the cap edge 2 and/or a behavior detection component 112 arranged outside the cap body or on the cap edge. The physiological detection component 111 can comprise at least one of a sound sensor, a heart rate detector, a blood pressure sensor, a body temperature sensor and a brain scanner. The behavior detection component 112 can comprise at least one of a camera and a limb sensor. For example, the camera 3 as shown in FIG. 4, where the camera 3 is arranged on the cap edge 2 and can capture the face of the user.

The execution component 13 of the emotion regulation device 10 can comprise at least one of a sound recording device, a wireless transmitting device, a sound alarm and a music player arranged within the cap body 1. The music player has earphones 5 arranged outside the cap body 1.

The execution component 13 of the emotion regulation device 10 can comprise smart glasses 6 with playing function arranged on the cap edge 2.

The execution component 13 of the emotion regulation device 10 can comprise at least one of an image recording device, a projector device and a signal indicator lamp arranged outside the cap body 1. For example, the image recording device 4 as shown in FIG. 4.

The emotion regulation device 10 can further comprise a storage component 14 and a power supply component 15 (the storage component 14 and the power supply component 15 are not shown in FIG. 4) arranged within the cap body 1.

Apparently, the skilled person in the art can make various modifications and variations to the present disclosure without departing from the spirit and the scope of the present disclosure. In this way, provided that these modifications and variations of the present disclosure belong to the scopes of the claims of the present disclosure and the equivalent technologies thereof, the present disclosure also intends to encompass these modifications and variations.

1. An emotion regulation device comprising:
at least one detection component for obtaining a current physiological parameter value and/behavior data of a user in real time;
a processing component or determining a current emotional state of the user based on the current physiological parameter value and/or the behaviour data, selecting an appropriate execution component based on the current emotional state, and sending a control instruction to the selected execution component; and
at least one execution component for executing a corresponding control action based on the control instruction.

2. The emotion regulation device as claimed in claim 1, wherein the detection component is used for: obtaining a parameter value for at least one physiological activity of voice, heart rate, blood pressure, body temperature and brainwave of a user as the current physiological parameter value, and/or obtaining data for at least one behavior of facial expression, gesture and pace of the user as the bevavior data.

3. The emotion regulation device as claimed in claim 2, wherein the detection component comprises a physiological detection component and/or a behavior detection component, the physiological detection component comprising at least one of a sound sensor, a heart rate detector, a blood pressure sensor, a body temperature sensor and a brain scanner, and the behavior detection component comprising at least one of a camera and a limb sensor.

4. The emotion regulation device as claimed in claim 1, wherein the processing component is used for: analyzing the current physiological parameter value and/or the behavior data, and determining one of a happy state, a mild anxiety state, a moderate anxiety state, a mild depressive state and a moderate depressive state as the current emotional state, wherein,
after the current emotional state is determined as the happy state, an execution component for recording is selected and a control instruction for recording an object, a sound and a scene that the user contacts is sent to the selected execution component for recording; and
after the current emotional state is determined as the mild anxiety state or the mild depressive state, an execution component for relieving emotion is selected and a control instruction for relieving user emotion is sent to the selected execution component for relieving emotion; and
after the current emotional state is determined as the moderate anxiety state or the moderate depressive state, an execution component for relieving emotion and an execution component for alarming are selected, and a control instruction for relieving user emotion is sent to the selected execution component for relieving emotion and a control instruction for alarming is sent to the selected execution component for alarming.

5. The emotion regulation device as claimed in claim 4, wherein the processing component is further used for, after the current emotional state of the user is determined as the mild anxiety state and does not change after a duration greater than or equal to a first time threshold, selecting an execution component for alarming and sending a control instruction for alarming to the selected execution component for alarming.

6. The emotion regulation device as claimed in claim 4, wherein the processing component is further used for, after the current emotional state of the user is determined as the mild depressive state and does not change after a duration greater than or equal to a second time threshold, selecting an execution component for alarming and sending a control instruction for alarming to the selected execution component for alarming.

7. The emotion regulation device as claimed in claim 4, wherein,
the execution component for recording comprises a sound recording device and/or an image recording device;
the execution component for relieving emotion comprises at least one of a music player with earphones, smart glasses capable of playing videos, a display device, arid a projector device; and
the execution component for alarming comprises at least one of a wireless transmitting device, a sound alarm, and a signal indicator lamp.

8. The emotion regulation device as claimed in claim 4, wherein the processing component is further used for sending the object, sound and scene that the user contacts when the user emotional state is the happy state recorded by the execution component for recording to the execution component for relieving emotion.

9. The emotion regulation device as claimed claim 4, wherein the emotion regulation device further comprises:
a storage component for storing the current physiological parameter value and/or the behavior data of the tier obtained by the detection component;
a power supply component for supplying power for the processing component, the detection component, the execution component and the storage component.

10. The emotion regulation device as claimed in claim 9, wherein the storage component is further used for storing the object, sound and scene that the user contacts when the the user emotional state is the happy state recorded by the execution component for recording; and
the execution component for relieving emotion is further used for calling the object, sound and scene that the user contacts when the user emotional state is the happy state.

11. A wearable device, comprising:
a device body;
an external accessory connected with the device body; and
the emotion regulation device as claimed in claim 1,

12. The device as claimed in claim 11, wherein the detection component, the processing component and the execution component are selectively arranged within the device body and on the external accessory.

13. The device as claimed in claim 11, wherein the emotion regulation device further comprises a storage component and a power supply component, the storage component and the power supply component being arranged within the device body.

14. A cap for relieving emotion, comprising:
a cap body;
a cap edge; and
the emotion regulation device as claimed in claim 1.

15. The cap as claimed in claim 14, wherein the detection component comprises a physiological detection component arranged within the cap body or on the cap edge and/or a behavior detection component arranged outside the cap body or on the cap edge, the physiological detection component comprising at least one of a sound sensor, a heart rate detector, a blood pressure sensor, a body temperature sensor and a brain scanner, and the behavior detection component comprising at least one of a camera and a limb sensor.

16. The cap as claimed in claim 14, wherein the execution component comprises:
at least one of a sound recording device, a wireless transmitting device a sound alarm and a music player arranged within the cap body, wherein the music player has earphones arranged outside the cap body; and/or
smart glasses with playing function arranged on the cap edge; and/or
at least one of an image recording device, a projector device and a signal indicator lamp arranged outside the cap body.

17. The cap as claimed in claim 14, wherein the emotion regulation device further comprises a storage component and a power supply component arranged within the cap body.

18. The device as claimed in claim 11, wherein the detection component is used for: obtaining a parameter value for at least one physiological activity of voice, heart rate, blood pressure body temperature and brainwave of a user as the current physiological parameter value, and/or obtaining data for at least one behavior of facial expression, gesture and pace of the user as the bevavior data.

19. The device as claimed in claim 11, wherein the processing component is used for: analyzing the current physiological parameter value and/or the behavior data, and determining, one of a happy state, a mild anxiety state, a moderate anxiety state, a mild depressive state and a moderate depressive state as the current emotional state, wherein,
after the current emotional state is determined as the happy state, an execution component for recording is selected and a control instruction for recording an object, a sound and a scene that the user contacts is sent to the selected execution component for recording; and
after the current emotional state is determined as the mild anxiety state or the mild depressive state, an execution component for relieving emotion is selected and a control instruction for relieving user emotion is sent to the selected execution component for relieving emotion; and
after the current emotional state is determined as the moderate anxiety state or the moderate depressive state, an execution component for relieving emotion and an execution component for alarming are selected, and a control instruction for relieving user emotion is sent to the selected execution component for relieving emotion and a control instruction for alarming is sent to the selected execution component for alarming.

20. The cap as claimed in claim 14, wherein the detection component is used for: obtaining a parameter value for at least one physiological activity of voice, heart rate, blood pressure, body temperature and brainwave of a user as the current physiological parameter value, and/or obtaining data for at least one behavior of facial expression, gesture and pace of the user as the bevavior data.
